(19) United States
(12) Patent Application Publication
(10) Pub. No.: US 2024/0245599 A1
(43) Pub. Date: Jul. 25, 2024

(54) Therapeutic Aftershave Skin Treatment Composition

(71) Applicant: Melwyn Turner, Port Wentworth, GA (US)

(72) Inventor: Melwyn Turner, Port Wentworth, GA (US)

(21) Appl. No.: 18/409,906

(22) Filed: Jan. 11, 2024

Related U.S. Application Data
(60) Provisional application No. 63/440,503, filed on Jan. 23, 2023.

(51) Int. Cl. A61K 8/9794; A61K 8/22; A61K 8/34; A61K 8/365; A61K 8/41; A61K 8/9789; A61Q 19/00

(52) U.S. Cl. CPC A61K 8/9794; A61K 8/22; A61K 8/345; A61K 8/365; A61K 8/41; A61K 8/9789; A61Q 19/00; A61Q 19/002

(57) ABSTRACT

An aftershave composition in the form of a lotion or cream for preventing razor burns, irritation, and skin dehydration caused due to shaving. The composition is easy to use and contains natural and organic components. The composition includes water, ethanol, aloe vera, fragrance, polyethylene glycol, tea tree oil, citric acid, triethanolamine, and hydrogen peroxide. The composition can be applied to any area of the body after a shave for moisturizing and hydrating skin and preventing razor burns. The composition can be used by individuals of all genders.

### CROSS-REFERENCE TO RELATED APPLICATION

The present application claims priority to, and the benefit of, U.S. Provisional Application No. 63/440,503, which was filed on Jan. 23, 2023, and is incorporated herein by reference in its entirety.

### FIELD OF THE INVENTION

The present invention generally relates to the field of skincare products. More specifically, the present invention relates to a novel aftershave composition for preventing razor burns, blemishes, skin irritations, and skin dehydration caused due to shaving of hair at different parts of the body. The aftershave lotion has a composition combining water-based ingredients and oil-based ingredients. The composition can be in the form of a cream, ointment, or lotion and can be easily applied on any part of the body by individuals to get smooth and hydrated skin without any skin irritation. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals shave various areas of their body including, but not limited to, the face, legs, underarms, and bikini lines to remove unwanted hair for both aesthetic and practical reasons. Shaving is a quick and relatively affordable method of hair removal that provides smooth skin temporarily. Individuals use different types of razors for shaving hair and shaving, while effective in hair removal, can lead to several issues. Firstly, shaving may cause razor burns and can irritate the skin, causing redness, itchiness, and discomfort. Skin irritation can be caused by a variety of factors, including using a dull razor, shaving too quickly, or using improper shaving techniques. Shaving may also lead to the development of blemishes, such as razor bumps or ingrown hairs. Blemishes occur when cut hairs curl back into the skin, causing inflammation and redness. Shaving may strip the skin of natural oils and moisture. This causes dry, rough, and dehydrated skin, especially if proper skin care is not followed post-shaving.

Many individuals try good quality shaving creams or gels to create a lubricated surface for shaving to reduce friction between the razor and the skin. Also, individuals use cold press and different lotions to moisturize their skin after shaving. Many individuals take warm showers or baths to soften the skin after shaving for easy moisturization. However, even after using such methods, individuals feel uncomfortable and disappointed with dry, rough, and blemished skin. Further, individuals use different lotions for different parts of the body for moisturization after shaving which is costly and time-consuming. Conventional lotions take more time to absorb into the skin which can delay the moisturization process. Individuals desire a more organic and effective solution for preventing dry and irritated skin after a shave.

Therefore, there exists a long-felt need in the art for an aftershave lotion or ointment for providing enhanced protection against irritation and blemishes after shaving. There is also a long-felt need in the art for an improved aftershave lotion that prevents shaving bumps on the skin. Additionally, there is a long-felt need in the art for an aftershave lotion that can be topically applied to skin after a shave for moisturization. Moreover, there is a long-felt need in the art for an aftershave lotion that is easily absorbed into the skin and prevents dehydration and roughness of skin after a shave. Further, there is a long-felt need in the art of a skin ointment that provides a soft, smooth, comfortable, and pleasant skin after a shave. Furthermore, there is a long-felt need in the art for an improved aftershave skin lotion that is transparent and can be applied to different parts of the body. Finally, there is a long-felt need in the art for an improved aftershave skin cream that offers a simple and convenient cream infused with a pleasant scent and can be applied to any area of the body to improve skincare routines, especially useful after shaving.

The subject matter disclosed and claimed herein, in one embodiment thereof, comprises an aftershave composition for preventing razor bumps, skin blemishes, and skin irritation after a shave. The composition includes ethanol, aloe vera extract, infused fragrance, polyethylene glycol, tea tree oil, citric acid, triethanolamine, hydrogen peroxide, and purified water as a carrier base, wherein the composition is in a select one of the following forms: a lotion, a cream, and a tonic. The composition when applied to skin enhances protection against irritation and blemishes after shaving. The composition also prevents shaving bumps on the skin and maximizes comfort while offering a pleasant fragrance.

In this manner, the novel therapeutic aftershave composition of the present invention accomplishes all of the foregoing objectives and provides users with a relatively safe, easy, cost-effective, and effective solution for preventing skin irritation, blemishes, razor bumps, and skin dehydration caused due to shaving hair on different body parts. The composition comes in different forms such as lotions and creams and can be topically applied on the shaved area. The composition improves skincare routines easily and effectively.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an aftershave composition for preventing razor bumps, skin blemishes, and skin irritation after a shave. The composition further comprises ethanol, aloe vera extract, infused fragrance, polyethylene glycol, tea tree oil, citric acid, triethanolamine, hydrogen peroxide, and purified water as a carrier base, wherein the composition is in a select one of the following forms: a lotion, a cream, and a tonic. The composition can be used by individuals of all ages and genders and can be applied to any part of the skin.

In a further embodiment of the present invention, a method of moisturizing skin after a shave of both men and women for preventing shaving bumps is disclosed and comprises the steps of contacting an individual's skin where moisturization is desired with an aftershave composition comprising: 5-8% ethanol, 5-7% aloe vera extract, 1-3% fragrant oil, 3-5% polyethylene glycol, 1-2% tea tree oil, 0.1-0.5% citric acid, 0.8-1.2% triethanolamine, 0.3-0.7% hydrogen peroxide, and 70-80% purified water.

In a further embodiment of the present invention, a composition of use as an aftershave transparent lotion comprises, by weight of the composition, 1-4% ethanol, 6-9% aloe vera extract, 2-4% essential oil, 1-3% polyethylene glycol, 0.1-2.3% tea tree oil, 0.4-1.5% citric acid, 0.3-0.9% triethanolamine, 0.1-0.4% hydrogen peroxide, 0.4-0.8% Phenoxyethanol, and 75-80% purified water.

The composition of the present invention does not require animal testing and contains the majority of natural and organic components. The composition is preferably transparent, readily absorbed in the skin, and helps in healing skin naturally.

The advantage of the aftershave composition of the present invention is that it provides users with an improved aftershave product designed to provide enhanced protection against irritation and blemishes after shaving and also prevents razor bumps. The ingredients act in harmony with each other to achieve the desired results of moisturizing skin. The lotion can be topically applied at room temperature and provides relaxation, cooling, and moisture to the skin surface after shaving. A user can apply a small amount of the composition on different body parts.

Numerous benefits and advantages of this invention will become apparent to those skilled in the art to which it pertains upon reading and understanding the following detailed specifications.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a diagrammatic representation of the components of the aftershave moisturizer lotion of the present invention used to prevent razor bumps and skin irritation;

FIG. 2 illustrates one potential embodiment of an aftershave lotion storing container, as per an exemplary embodiment;

FIG. 3 illustrates another embodiment of the container in which the aftershave composition can be available for consumers; and

FIG. 4 illustrates a flow chart showing steps used in manufacturing therapeutic aftershave skin treatment composition of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an aftershave lotion or ointment for providing enhanced protection against irritation and blemishes after shaving. There is also a long-felt need in the art for an improved aftershave lotion that prevents shaving bumps on the skin. Additionally, there is a long-felt need in the art for an aftershave lotion that can be topically applied to skin after a shave for moisturization. Moreover, there is a long-felt need in the art for an aftershave lotion that is easily absorbed into the skin and prevents dehydration and roughness of skin after a shave. Further, there is a long-felt need in the art of a skin ointment that provides for soft, smooth, comfortable, and pleasant skin after a shave. Furthermore, there is a long-felt need in the art for an improved aftershave skin lotion that is transparent and can be applied to different parts of the body. Finally, there is a long-felt need in the art for an improved aftershave skin cream that offers a simple and convenient cream infused with a pleasant scent and can be applied to any area of the body to improve skincare routines, especially useful after shaving.

The present invention, in one exemplary embodiment, is a method of moisturizing skin after a shave of both men and women for preventing shaving bumps. The method includes the steps of contacting an individual's skin where moisturization is desired with an aftershave composition comprising: 5-8% ethanol, 5-7% aloe vera extract, 1-3% fragrant oil, 3-5% polyethylene glycol, 1-2% tea tree oil, 0.1-0.5% citric acid, 0.8-1.2% triethanolamine, 0.3-0.7% hydrogen peroxide and 70-80% purified water.

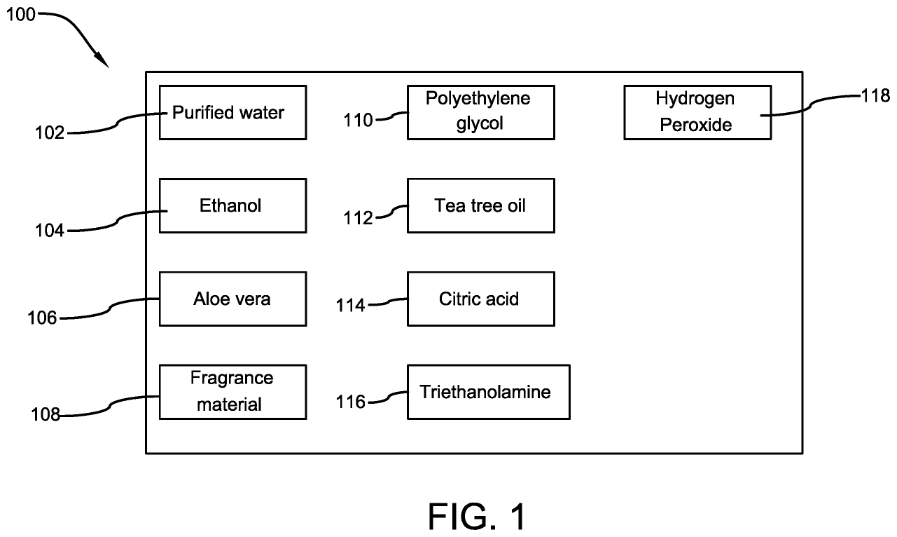

Referring initially to the drawings, FIG. 1 illustrates a diagrammatic representation of the components of the aftershave moisturizer lotion of the present invention used to prevent razor bumps and skin irritation. The aftershave moisturizer lotion 100 of the present invention is made up of a majority of organic ingredients which can be derived from natural or commercial sources. All the ingredients provide a unique quality and increase the effectiveness of the aftershave moisturizer lotion 100 in preventing razor bumps, skin irritation, and blemishes caused due to shaving hair on different parts of the body.

Purified water 102 can be in the form of distilled water and forms the base for the aftershave skin treatment cream 100. The water 102 helps in even distribution of other ingredients in the lotion and helps in maintaining skin hydration as well. Ethanol 104 provides antiseptic properties and helps to disinfect the skin and prevent infection in the case of small nicks or cuts that may occur during shaving. Ethanol helps to kill bacteria and other microorganisms that can cause infections. Further, ethanol 104 has astringent properties and helps to reduce inflammation. Rapid evaporation of ethanol 104 helps to cool the skin and soothe irritation.

Aloe vera extract 106 can be included in the lotion 100 in the form of a gel or a powder. Aloe vera extract 106 has natural anti-inflammatory and antibacterial properties, which can help to soothe irritated skin and prevent infections. Further, aloe vera 106 is a humectant and helps keep the skin hydrated while providing a cooling effect to soothe irritation. Aloe vera 106 contains antioxidants, such as vitamins C and E, which help protect the skin from free radical damage caused by environmental factors. Aloe vera 106 can be extracted directly from the Aloe vera plant or any commercially available aloe vera extracts.

Fragrance material 108 in the form of natural essential oils is infused in the lotion composition 100. The fragrance material 108 provides a pleasant scent to the lotion 100 and masks any chemical odors from other ingredients to enhance the user's experience. Different essential oils such as rosemary oil, jojoba oil, almond oil, and more can be used for providing fragrance to the lotion 100. To meet the requirements of different users, herbal extracts, derived from plants such as mint, eucalyptus, or thyme, offering herbal or medicinal scents can be used in the aftershave lotion product 100.

Polyethylene glycol (PEG) 110 is a natural moisturizer and helps in softening the skin. PEG 110 is a synthetic polymer made up of repeating units of ethylene glycol and is a clear, colorless, and odorless liquid that is soluble in water and alcohol. PEG is a humectant and helps to attract water to the skin. Further, PEG 110 used in the lotion 100 can have a molecular weight from about 200 to about 800.

Tea tree oil 112 is a natural essential oil and is extracted from the leaves of the Melaleuca alternifolia plant to be included in the lotion 100. Tea tree oil 112 has antimicrobial properties against a variety of bacteria, fungi, and viruses and helps to prevent infections and reduce the risk of razor burn. Further, tea tree oil 112 is a humectant and helps to attract water to the skin.

Citric acid 114 helps in maintaining an acidic pH of the lotion composition 100 to help maintain the pH of the skin after shaving. Further, citric acid 114 has astringent properties to reduce inflammation. Also, citric acid has a faint, citrusy odor which adds to the scent of the lotion composition 100.

Triethanolamine (TEA) 116 has emulsifying, buffering, and moisturizing properties. More specifically, TEA 116 helps to combine oil and water which makes the aftershave lotion 100 both moisturizing and non-greasy. TEA 116 is slightly basic and has a pH of around 8, which prevents the lotion 100 from becoming too acidic.

Hydrogen peroxide 118 functions as a disinfectant and helps to kill bacteria and other microorganisms that can cause infections. Further, hydrogen peroxide 118 automatically cleans and disinfects cuts caused by a razor during shaving. It will be apparent to a person skilled in the art that hydrogen peroxide 118 is always less than 1% in weight in the therapeutic aftershave skin treatment lotion 100 to avoid skin irritation. In some embodiments, Phenoxyethanol is also included as a preservative. Phenoxyethanol is a synthetic preservative and prevents the growth of bacteria and mold.

The aftershave lotion composition of the present invention is safe for humans and has been demonstrated to not cause irritation to the skin even when used multiple times in a day and for a long period of time.

Referring to Table 1, there is disclosed an aftershave lotion composition with a number of different components in varying quantities in order to achieve a desired result. The lotion product 100, in one potential embodiment, is provided as a transparent lotion. One such exemplary embodiment of the aftershave lotion composition 100 is provided as shown in Table 1.

TABLE 1

| | Composition in percentage |
|---|---|
| Component | by weight (% w/w) |
| Ethanol | 5-8 |
| Aloe vera extract | 5-7 |
| Fragrance material | 1-3 |
| Polyethylene Glycol | 3-5 |
| Tea Tree Oil | 1-2 |
| Citric Acid | 0.1-0.5 |
| Triethanolamine | 0.8-1.2 |
| Hydrogen Peroxide | 0.3-0.7 |
| Purified water | An amount to make the total 100 |

Table 2, there is disclosed alternative aftershave lotion product 100 with a number of different components in varying quantities in order to achieve a desired result.

TABLE 2

| | Composition in percentage |
|---|---|
| Component | by weight (% w/w) |
| Ethanol or Isopropyl alcohol | 1-4 |
| Aloe vera extract | 6-9 |
| Fragrance material | 2-4 |
| Polyethylene Glycol | 1-3 |
| Tea Tree Oil | 0.1-2.3 |
| Citric Acid | 0.4-1.5 |
| Triethanolamine | 0.3-0.9 |
| Hydrogen Peroxide | 0.1-0.4 |
| Phenoxyethanol | 0.4-0.8 |
| Purified water | An amount to make the total 100 |

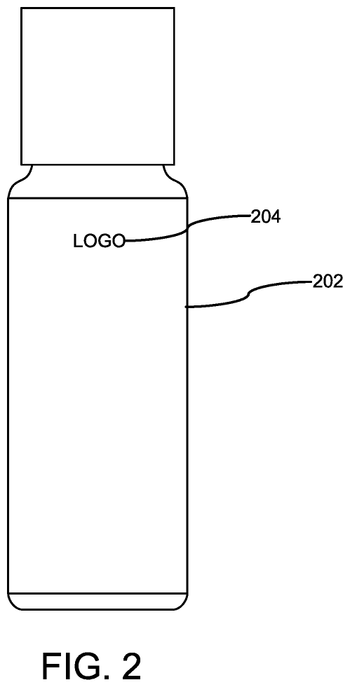

FIG. 2 illustrates one potential embodiment of an aftershave lotion storing container, as per an exemplary embodiment. As shown, the lotion 100 is stored in the container 202 for easy accessibility and portability. The container 202 can be of different sizes ranging from 8 oz. to 40 oz. to meet the requirements of different users. Further, the container 202 may be of any suitable shape and size, including, but not limited to, rectangular, cubic, pyramidal, cylindrical, spherical, or any other shape. The container 202 can also include a logo, indicia, or trademark 204 along with customizable colors and fonts, embroidery and prints, and/or other images on the surface. A user can take the desired quantity of lotion from the container 202 and apply to the target skin area smoothly and uniformly. In different embodiments, the aftershave lotion can be taken out in the form of a mist or in the form of a thick liquid.

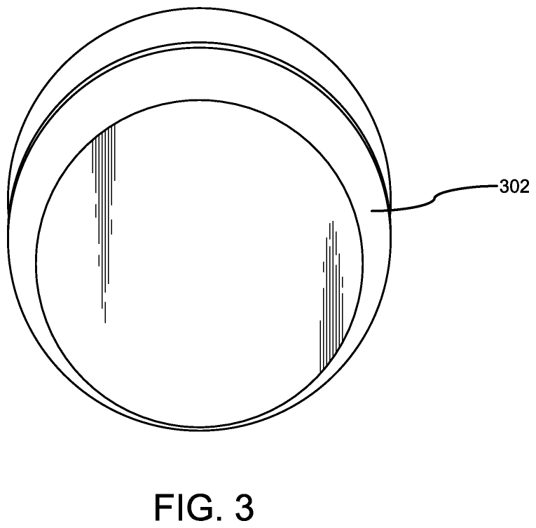

FIG. 3 illustrates another embodiment of the container in which the aftershave composition can be available for consumers. In the present embodiment, the aftershave composition is in the form of an ointment or cream and is stored in the container 302. The aftershave composition 100 can be configured to have sunscreen properties with a rating up to SPF 50 for providing protection from UV rays of sunlight.

The composition of the present invention is useful as a pharmaceutical or an ayurvedic product and functions for all genders.

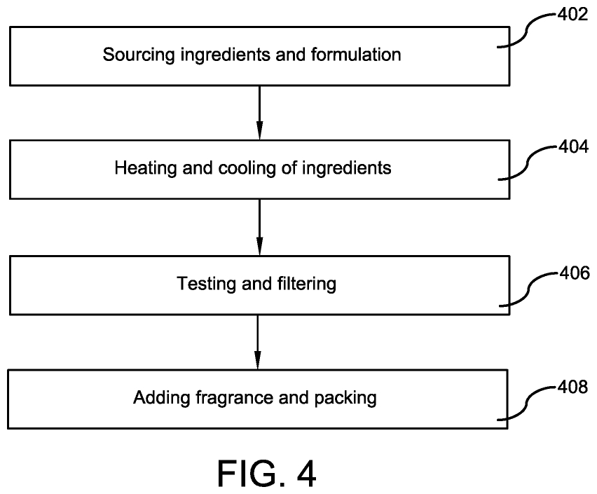

FIG. 4 illustrates a flow chart showing steps used in manufacturing therapeutic aftershave skin treatment composition of the present invention in accordance with the disclosed architecture. Initially, ingredients described in Tables 1 and 2 are sourced from natural or commercial sources and the desired formulation is formed as per manufacturing requirements (Step 402). Then, all the ingredients are mixed and heated in a vacuum chamber to prevent oxidation. Repeated and quick heating and cooling are performed for the incorporation of the therapeutic properties of different ingredients while maintaining a liquid nature of the mixture (Step 404). In the next step 406, different testing techniques such as microbiological testing, sterilization, and filtering to remove any remains of the ingredients are performed. The testing is performed to maintain the purity of the aftershave composition. Finally, additional fragrance and aroma are added to create different profiles of the composition and the composition is stored and packed in different containers (Step 408).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “aftershave skin treatment cream”, “aftershave moisturizer lotion”, “therapeutic aftershave skin treatment lotion”, “aftershave lotion”, “lotion product”, and “lotion” are interchangeable and refer to the therapeutic aftershave skin treatment composition 100 of the present invention.

Notwithstanding the foregoing, the therapeutic aftershave skin treatment composition 100 of the present invention can contain any additional ingredient known in the art without affecting the overall concept of the invention, provided that it accomplishes the above-stated objectives. One of ordinary skill in the art will appreciate that the ingredients and composition of the therapeutic aftershave skin treatment composition 100 as shown in the FIGS. are for illustrative purposes only, and that many other compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A moisturizer lotion comprising:
a moisturizer lotion composition comprising an ethanol, an aloe vera extract, a fragrant oil, a polyethylene glycol, a tea tree oil, a citric acid, a triethanolamine, a hydrogen peroxide, and a purified water.

2. The moisturizer lotion of claim 1, wherein a weight of said ethanol is from 5% to 8% of a weight of said moisturizer lotion composition.

3. The moisturizer lotion of claim 2, wherein a weight of said aloe vera extract is from 5% to 7% of said weight of said moisturizer lotion composition.

4. The moisturizer lotion of claim 3, wherein a weight of said fragrant oil is from 1% to 3% of said weight of said moisturizer lotion composition.

5. The moisturizer lotion of claim 4, wherein a weight of said polyethylene glycol is from 3% to 5% of said weight of said moisturizer lotion composition.

6. The moisturizer lotion of claim 5, wherein a weight of said tea tree oil is from 1% to 2% of said weight of said moisturizer lotion composition.

7. The moisturizer lotion of claim 6, wherein a weight of said citric acid is from 0.1% to 0.5% of said weight of said moisturizer lotion composition.

8. The moisturizer lotion of claim 7, wherein a weight of said triethanolamine is from 0.8% to 1.2% of said weight of said moisturizer lotion composition.

9. The moisturizer lotion of claim 8, wherein a weight of said hydrogen peroxide is from 0.3% to 0.7% of said weight of said moisturizer lotion composition.

10. The moisturizer lotion of claim 9, wherein a weight of said purified water is a remaining balance of a weight of said moisturizer lotion composition.

11. An aftershave moisturizer comprising:
an aftershave moisturizer composition comprising an ethanol having a weight from 1% to 4%, an aloe vera extract having a weight from 6% to 9%, a fragrant oil having a weight from 2% to 4%, a polyethylene glycol having a weight from 1% to 3%, a tea tree oil having a weight from 0.1% to 2.3%, a citric acid having a weight from 0.4% to 1.5%, a triethanolamine having a weight from 0.3% to 0.9%, a hydrogen peroxide having a weight from 0.1% to 0.4%, a phenoxyethanol having a weight from 0.4% to 0.8%, and a remainder of a purified water of a composition weight.

12. The aftershave moisturizer of claim 11, wherein said aftershave moisturizer is selected from the group consisting of a mist, a liquid, an ointment, and a cream.

13. The aftershave moisturizer of claim 12, wherein said fragrant oil is selected from the group consisting of a rosemary oil, a jojoba oil, and an almond oil.

14. The aftershave moisturizer of claim 12, wherein said fragrant oil is selected from the group consisting of a mint, a eucalyptus, and a thyme.

15. An aftershave moisturizer comprising:
an aftershave moisturizer composition comprising an ethanol having a weight from 5% to 8%, an aloe vera extract having a weight from 5% to 7%, a fragrant oil having a weight from 1% to 3%, a polyethylene glycol having a weight from 3% to 5%, a tea tree oil having a weight from 1% to 2%, a citric acid having a weight from 0.1% to 0.5%, a triethanolamine having a weight from 0.8% to 1.2%, a hydrogen peroxide having a weight from 0.3% to 0.7%, and a remainder of purified water of a composition weight.

16. The aftershave moisturizer of claim 15 further comprising a phenoxyethanol having a weight from 0.4% to 0.8%.

17. The aftershave moisturizer of claim 15, wherein said aftershave moisturizer is selected from the group consisting of a mist, a liquid, an ointment, and a cream.

18. The aftershave moisturizer of claim 17, wherein said fragrant oil is selected from the group consisting of a rosemary oil, a jojoba oil, and an almond oil.

19. The aftershave moisturizer of claim 18, wherein said tea tree oil is a natural essential oil extracted from the leaves of the Melaleuca alternifolia plant.

20. The aftershave moisturizer of claim 19, wherein said purified water is distilled water.
